(19) United States
(12) Patent Application Publication
(10) Pub. No.: US 2002/0132236 A1
(43) Pub. Date: Sep. 19, 2002

(54) Specific inhibitors of ribosome recycling factors (RRF)

(76) Inventors: Hideko Kaji, Jenkintown, PA (US); Akira Kaji, Jenkintown, PA (US)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 09/824,304

(22) Filed: Apr. 2, 2001

Related U.S. Application Data
(60) Provisional application No. 60/195,149, filed on Apr. 6, 2000.

(51) Int. Cl. A61K 48/00; C12Q 1/68; C07H 21/02

(52) U.S. Cl. 435/6; 536/23.1; 514/44

(57) ABSTRACT

Oligoguanylic acid inhibits the activity of Ribosome Recycling Factor (RRF), a soluble factor responsible for disassembly of the post-termination complex. RRF is essential for all bacteria because it is required for the recycling of spent ribosomes to initiate the translation of another mRNA into another polypeptide. As eukaryotic cytoplasmic protein synthesis does not require RRF, inhibitors of RRF will be efficacious as an anti-bacterial agent. The present invention provides oligoguanylic acid, sd-G4, or derivatives thereof, in particular, to serve as a lead compound for the development of novel anti-bacterial therapeutic agents.

### CROSS REFERENCE TO RELATED APPLICATIONS

This application claims priority in part under 35 U.S.C. §119 based upon U.S. Provisional Patent Application No. 60/195,149 filed Apr. 6, 2000.

### FIELD OF THE INVENTION

The present invention relates to the field of microbiology and pharmacology and to the use of oligoguanine as a lead compound in the identification of small molecules that inhibit Ribosome Recycling Factor (RRF) and, more particularly, to the incorporation of at least one of these compounds into pharmaceutical compositions for the development of anti-bacterial therapeutics.

### BACKGROUND OF THE INVENTION

RRF has been found to be a perfect mimic of tRNA (transfer RNA) in size and structure. It has been postulated that it mimics tRNA functionally (Selmer et al., 1999). RRF can bind to the A-site and P-site on the ribosomes, thus releasing MRNA and tRNA from the ribosomes. It is known that in the neighborhood of the A- and P-sites on ribosomes, guanine residues of ribosomal RNA are abundantly exposed and appear to interact with the ribosome bound RRF.

In addition, it has been found previously that G4 (tetra G) is an effective anti-HIV. (Fujihashi, T., et al., Biochemical & Biophysical Communications 203: 1224-1250, 1994; Fujihashi, T., et al., AIDS Research and Human Retroviruses 11: 4037-4044, 1995). One of the mechanisms associated with this inhibition by an oligoG (oligo guanylic acid) is a specific inhibition of reverse transcriptase (RT). The inhibition by tetraG could be on the RT-bound tRNA, which functions as an RT primer.

As has been repeatedly pointed out in several articles on RRF (Janosi, L., et al., Adv Biophys 32: 121-201, 1996; Janosi, L., et al., Biochimie 78: 959-969, 1996; Kaji, A., Hirokawa, G., “Ribosome recycling factor: An essential factor for protein synthesis.” The Ribosome: Structure, Function, Antibiotics, and Cellular Interactions. ASM Press, Washington D.C., 527-39, 2000; Kaji A., Teyssier E., et al., Biochem Biophys Res Commun 250: 1-4, 1998), RRF is an ideal target of new antibacterial agents. Inhibition of RRF results in the ribosome remaining bound to the MRNA, and thus unable to initiate synthesis of a new polypeptide strand. This has a bactericidal and/or bacteriostatic effect. Inhibition of RRF will not influence eukaryotic cytoplasmic protein synthesis, as the eukaryotic protein synthesis system does not have the equivalent of an RRF. One may expect that the proposed RRF inhibitor would be detrimental to respiration, as mitochondria do contain an RRF, but the inhibitory effects on mitochondrial protein synthesis could be minimized by the rational drug design of an RRF inhibitor using sd-G4, or derivatives thereof, as a lead compound. Certain of the antibiotics currently used to treat bacterial infections, such as erythromycin, tetracycline and chloramphenicol, inhibit mitochondrial protein synthesis in addition to bacterial protein synthesis. The wide use of these antibiotics implies that any side effects due to inhibition of on mitochondrial protein synthesis are limited.

Despite the fact that RRF is an ideal target for antibacterial agents, there have not been any lead compounds for development of anti-RRF agents. The available crystal structure will provide a structural basis for the development of lead compounds, which are vital for rational drug design. The present invention provides a lead compound for the development of novel therapeutic agents that have a bactericidal and/or bacteriostatic effect.

The present invention describes oligoG as is a specific inhibitor of RRF. As such, oligoG will serve as a lead compound in the discovery and development of anti-bacterial therapeutic agents. Lead compounds will be used to obtain an effective inhibitor of RRF using a rational drug design approach based on the recently available crystal structure of RRF. (Japanese patent pending Hei 11-158637). The present invention, also includes a method of discovering specific RRF inhibitors and to the use of these anti-RRF compounds for the treatment of bacterial infections.

### DEFINITIONS

“sd-G4” means “phosphorothioate tetradeoxyguanylic acid”

“polysome” means “polyribosome”

“monosome” means “ribosome”

### SUMMARY OF THE INVENTION

The present invention provides a method of identifying an inhibitor of RRF. The inhibitor to be tested is mixed in a reaction that includes RRF and polysomes. The conversion of the polysome to monosomes is measured and an inhibitor(s) is identified by a decrease in the conversion of the polysome to the monosomes. Confirmation that the inhibitor inhibits translation of natural MRNA and establishment that the inhibitor does not inhibit translation of a synthetic polynucleotide is further determined. In one embodiment the inhibitor is sd-G4, or derivatives thereof.

It is a further object of the invention to provide an inhibitor of RRF wherein the inhibitor is a small molecule that is structurally and functionally related to sd-G4, or derivatives thereof, with improved properties that inhibit RRF, thereby inhibiting disassembly of a post-termination complex and thus protein synthesis. In one embodiment the inhibitor has a ribose moiety included in the small molecule that is structurally and functionally related to sd-G4, or derivatives thereof.

It is also an object of the present invention to provide an inhibitor of RRF, wherein sd-G4, or derivatives thereof, is used as a lead compound for further development of a therapeutic agent that inhibits RRF activity.

It is an object of the present invention to provide a method of identifying an inhibitor of RRF, wherein sd-G4, or derivatives thereof, is used as a lead compound. The affinity of a set of ligands is screened to identify a set of ligands. Each of the ligands is mixed in a reaction including RRF and polysomes and the conversion of the polysome into monosmes is measured. The inhibitor is identified by a decrease in the conversion of the polysome into monosomes Confirmation that the inhibitor inhibits translation of natural mRNA and establishment that the inhibitor does not inhibit translation of a synthetic polynucleotide is further determined. In one embodiment the inhibitor is a small molecule, the small molecule being structurally and functionally related to the lead compound (sd-G4, or derivatives thereof) with improved properties that inhibit RRF, thereby inhibiting disassembly of a post-termination complex and thus protein synthesis. In one embodiment the dissassembly of the post-termination complex and protein synthesis results in a bactericidal and/or bacteriostatic effect.

The present invention also provides a pharmaceutical composition, which includes a pharmaceutically acceptable carrier and a therapeutically effective amount of an inhibitor of RRF.

### BRIEF DESCRIPTION OF FIGURES

FIG. 1: Inhibition of RRF by sd-G4. The conversion of polysomes to monosome (70S ribosome) is regarded as a function of RRF and EF-G. The results show that sd-G4 inhibits the RRF reaction in a dose-dependent manner. As low as 5 μM sd-G4 significantly inhibited the reaction.

FIG. 2 Excess RRF reduces inhibition by sd-G4. The reaction mixtures (275 μl) contained: (•) 1× RRF(72 ng/ml) or (∘) 2× RRF (144 ng/ml). The remaining components are the same as described (infra). The inhibition of RRF is determined as described (FIG. 1). Inhibition by sdg4 is reduced by addition of more RRF.

FIG. 3: sd-G4 inhibits MS2 RNA dependent protein synthesis. Translation of natural mRNA (MS2 phage RNA) into protein is clearly inhibited by sd-G4 (compare the filled circle with all other symbols). As low as 7 μM sd-G4 results in a 50% inhibition of protein synthesis (see the 8 minute point, and compare the filled triangle with the circle).

FIG. 4: sd-G4 does not inhibit the translation of polyuridylic acid (Poly U) into polyphenylalanine. The filled circle represents the complete system. It is clear that as high as 10 μM sd-G4 does not inhibit polyphenylalanine formation as measured by the incorporation of labeled phenylalanine into polyphenylalanine. In fact, for unknown reasons sd-G4 stimulated the synthesis of polyphenylalanine.

FIG. 5: Oligonucleotide specificity. The reaction mixture is as described (infra) except that various tetra oligonucleotides are added as indicated. The inhibitory effect of various oligonucleotides is expressed as a percent inhibition, as in FIG. 1. This dose response curve of the inhibitory activity of various oligophosphorothioate on the RRF reaction, reveals that only sd-G4 (open squares) has inhibitory activity, while all other homo oligophosphorothioate nucleotides have no activity.

FIG. 6: Longer chain length oligoG results in a more efficacious inhibitor. This dose dependent curve clearly indicates that among those tested, the most efficient inhibitor was sd-G8. In fact, the longer the chain, the more effective the inhibition.

FIG. 7: Phosphorothioate, but not phosphodiester oligoguanylic acid inhibits the RRF reaction. The reaction mixture (275 μl) for the RRF reaction is as described (infra), except that various amounts of sd-G4 and pd-G4 (phospo diester tetraguanylic acid) are added, as indicated. The RRF reaction is performed and the inhibitory effects of sd-G4 and pd-G4 are measured as in FIG. 1. The values are expressed as percent inhibition. It is clear from this dose response curve of sd-G4 for the RRF reaction (open circle) that only a phosphorothioate derivative is effective. The counterpart of sd-G4, the phosphodiester version of oligoguanylic acid (pd-G4, filled circle) is not effective in inhibiting RRF.

FIG. 8: sd-G4 does not inhibit release of ribosome-bound deacylated tRNA by RRF. The reaction mixture contains various amounts of E. coli polysome as shown. The amount of released tRNA is proportional to the amount of polysomes used. The important point of this figure is that the release of tRNA in the absence of sd-G4 is identical to the release in the presence of 7 μM sd-G4. This indicates that the inhibitor does not inhibit release of tRNA but inhibits the release of ribosomes from mRNA.

FIG. 9: Gel Retardation assay with E. coli RRF and sd-G4. It is clear that RRF binds to 32P-end-labeled sd-G4, as the electrophoretic movement of the sd-G4 is retarded (i.e; it's movement through the gel is slowed) in the presence of RRF (Lane 2). A negative control with BSA shows no interaction between the sd-G4 and RRF (Lane 3.) The interaction is inhibited by the addition of non-labeled sd-G4 (Lane 4, 5, 6, 7.) These data reveal the direct interaction between RRF and sd-G4.

### DETAILED DESCRIPTION OF THE INVENTION

Methods

Inhibition of RRF by sd-G4.

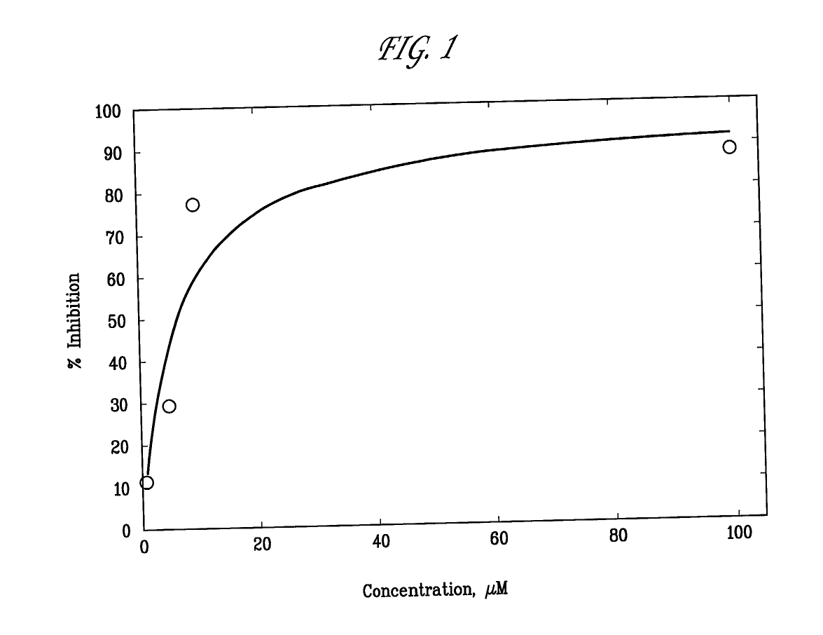

The reaction mixture (275 μl) contained 72 ng/ml RRF, 0.4 μg/ml EF-G, 363 nM GTP, 10 mM Tris-Cl, pH 7.4, 8.2 mM MgSO₄, 0.8M NH₄Cl, 0.3 mM DTT, 50 mM puromycin, 1 A₂₆₀ polysome of E. coli, and an increasing concentration of sd-G4, as indicated in FIG. 1. The reaction mixture is incubated for 15 min at 30° C., loaded onto a sucrose gradient (15% to 30% in buffer J (Hirashima, A., Kaji, A., Biochemistry 11: 461-471, 1972) and centrifuged at 40,000 rpm for 75 min at 4° C. The ISCO density gradient analyzer monitored sedimentation profiles of the polyribosome. The value is expressed as a percent inhibition calculated from the area of 70S and polysome on those profiles.

sd-G4 Inhibits MS2 RNA Dependent Protein Synthesis.

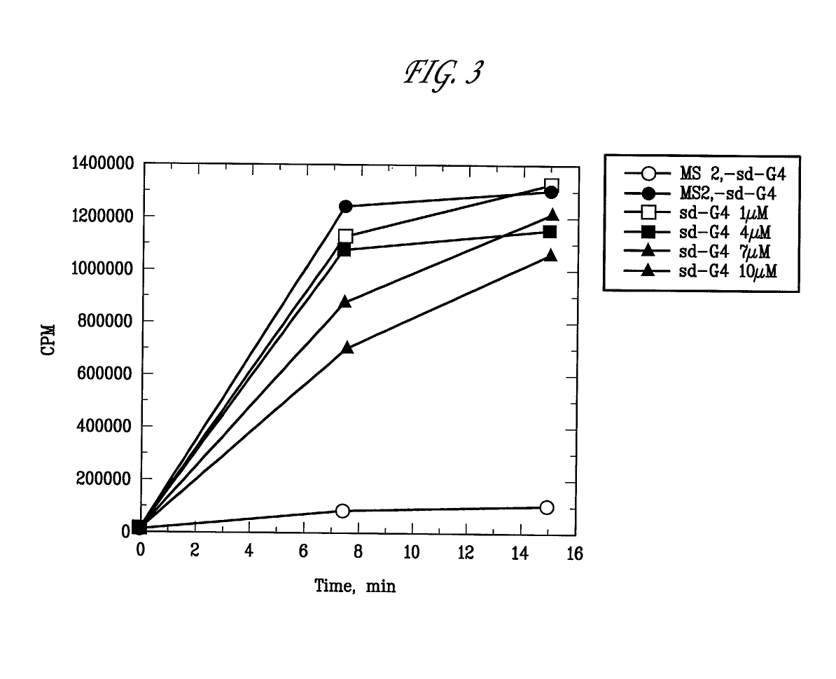

The reaction mixture (30 μl) contains 12 μl S30 (Kaji, 1968), Premixture (Promega), 2.4 μg MS2 phage RNA (Boehringer Mannheim), 0.1 mM amino acids mixture minus methionine (Promega), 10.1 pmole ³⁵S-methionine (NEN, 0.6 Ci/μmole), 0.6 A ²⁶⁰U S30 extract (Promega), and 1, 4, 7, or 10 μM sd-G4 (FIG. 3). The reaction mixture is incubated at 37° C. At various times after the onset of the reaction, hot TCA (trichloroacetic acid) insoluble counts are measured.

sd-G4 Does Not Inhibit Translation of Polyuridylic Acid (Poly U) into Polyphenylalanine.

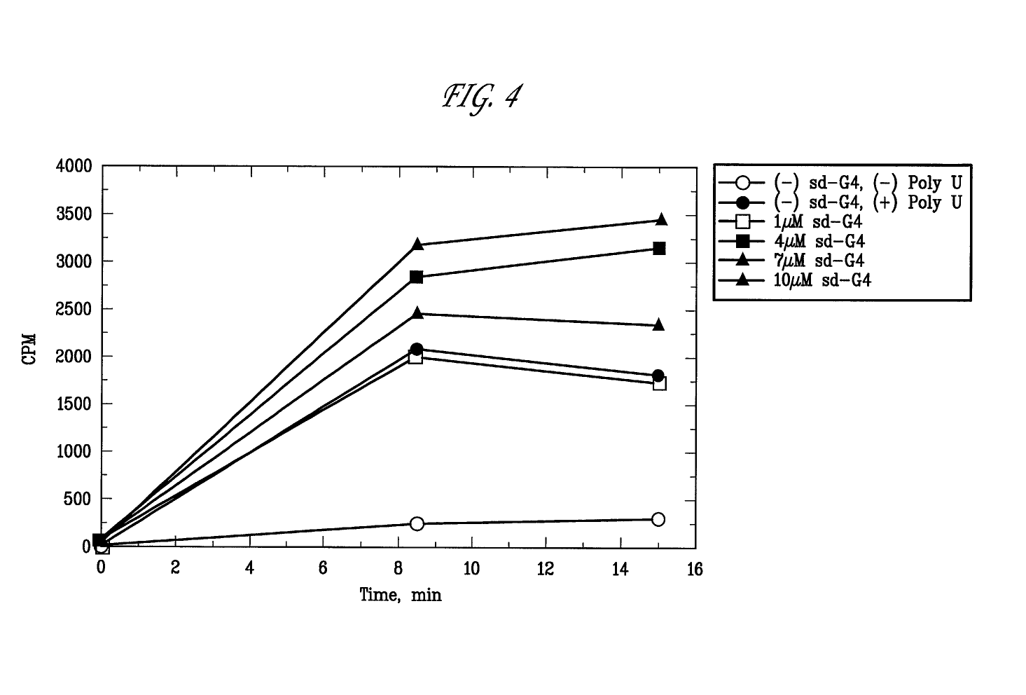

The reaction mixture (30 μl) contains 12 μl S30, Premixture (Promega), 20 μM ¹⁴C-phenylalanine (Amersham, 450 Ci/mole), 10 mM Mg acetate (MgAc), 0.08 μg/ml polyU (Sigma), 1 A₂₆₀ unit of E. coli S30 extract (Promega), and 1, 4, 7, or 10 μM sd-G4 (FIG. 4). The reaction mixture is incubated at 37° C. At various periods after the onset of the reaction, hot TCA (trichloroacetic acid) insoluble counts are measured.

sd-G4 Does Not Inhibit Release of Ribosome-Bound Deacylated tRNA by RRF.

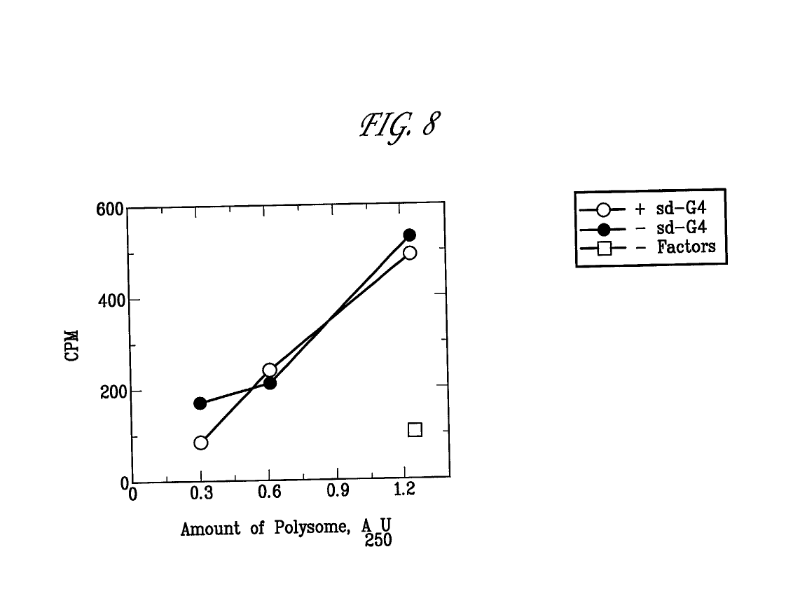

The reaction mixture (550 μl) for the RRF reaction contains 0.2 nmole RRF where indicated, 0.2 nmole EF-G, 0.37 μM GTP, 275 μM puromycin, 10 μM sd-G4 where indicated, and various amounts of E. coli polysome (see FIG. 8). The mixture is incubated for 15 min. at 30° C. The released tRNA is separated from the complex with pre-washed Microcon 100 (Millipore, 330× g, 40 min). The filter is washed once by centrifugation with 550 μl of a buffer (10 mM Tris-Cl, pH 7.6, 8.2 mM MgSO₄, 80 mM NH₄Cl, 0.14 mM DTT) and combined with the filtrate. Separated tRNA solution is concentrated with Microcon 30 (Millipore, 14,000× g, 15 min. twice) to a total volume of 14 μl. The concentrated tRNA is charged with 0.15 μCi of ¹⁴C-amino acids mixture (Amersham, 52 mCi/mg) in 30 μl of the reaction mixture (50 mM Tris-Cl, pH 7.8,10 mM MgAc, 6 mM β-mercaptoethanol, 3 mM ATP, 5 mM phosphoenolpyruvate, 138 μg pyruvate kinase, 33.3 μg aminoacyl tRNA synthetases). Cold (4° C.) TCA (trichloro acetic acid) insoluble radioactivity is measured to estimate the amount of tRNA released. In this experiment, the amount of tRNA released is measured by its capacity to accept an amino acid.

Gel Retardation Assay with E. coli RRF and sd-G4.

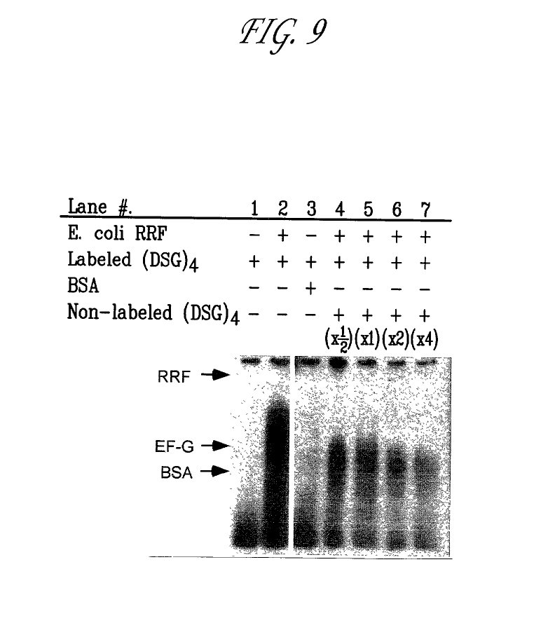

The reaction mixture for the binding of ³²P-end-labeled sd-G4 to RRF (10 μl) contains 8 mM Tris-Cl(pH 7.6), 9.7 mM MgSO₄, 81 mM NH₄Cl, 1.3 mM DTT, and approximately 2.4 μM of ³²P-end-labeled sd-G4. E. coli RRF, EF-G, polysome, BSA are added as indicated in FIG. 9 and competitor non-labeled sd-G4 is added at ½, 1, 2, and 4 times the concentration of the labeled sd-G4 (FIG. 9). The mixture is incubated at 30° C. for 15 min. 5% Glycerol and one-tenth volume of 25% Ficoll containing xylane cyanol and bromphenol blue is added, and the entire reaction mixture is subjected to 4% polyacrylamide gel (29:1, acrylamide/bisacrylamide) electrophoresis at 10 V/cm with constant recirculation of TBE buffer (89 mM Tris-borate, 2.5 mM EDTA, pH 8.3). The gel is dried onto Whatman 3MM paper and subject to autoradiography.

Toxicity Studies.

Swiss Webster male mice weighing 20-25 grams receive intraperitoneal or intravenous injections of tetra guanylic acid, dissolved in 0.9% sodium chloride, see Table 1 for dosages.

TABLE 1
Lack of Toxicity of sd-G4 on Mice

| | | Route | No. of mice | Visual |
|---|---|---|---|---|
| Compound | Dose/kg | of administration | used | effect |
| d-G4^{(*1)} | 125 mg | iv, ip | 3 each | none |
| sd-G4 | 20 mg-125 mg | iv, ip | 3 each | none |
| p sd-G4^{(*2)} | 20 mg-125 mg | iv, ip | 3 each | none |
| (RIP)₄^{(*3)} | 125mg | iv, ip | 3 each | none |

Compounds were dissolved in 0.9% NaCl
^{(*1)}deoxyguarylic acid
^{(*2)}5′ phosphorylated sd-G4
^{(*3)}riboinosinic tetraphosphate

Lead Compounds

The development of new small molecule therapeutics typically begins with the identification of a lead compound that exhibits some of the properties required for safe and effective therapeutic intervention. Compounds with improved properties are subsequently derived through iterative cycles of analog preparation and testing. Since all lead compounds share the common property that they must bind to their macromolecular targets, computer programs can be used to identify unoccupied (aqueous) space between the van der Waals surface of a compound and the surface defined by residues in the binding site. These gaps in atom-atom contact represent volume that could be occupied by new functional groups on a modified version of the lead compound, ie; sd-G4, or derivatives thereof. More efficient use of the unoccupied space in the binding site could lead to a stronger binding compound if the overall energy of such a change is favorable.

A region of the binding pocket that has unoccupied volume large enough to accommodate the volume of a group equal to or larger than a covalently bonded carbon atom can be identified as a promising position for functional group substitution. Functional group substitution at this region can constitute substituting something other than a carbon atom, such as oxygen. If the volume is large enough to accommodate a group larger than a carbon atom, a different functional group that would have a high likelihood of interacting with protein residues in this region may be chosen. Features which contribute to interaction with protein residues and identification of promising substitutions include, but are not limited to, hydrophobicity, size, rigidity and polarity. The combination of docking, Kᵢ estimation, and visual representation of sterically allowed room for improvement permits prediction of potent derivatives.

Screening by affinity identifies a small subset of ligands that are subsequently tested for function using a variety of biochemical assays. Assays of the invention are particularly amenable to high throughput screening (HTS) assays. HTS permits screening of large numbers (i.e., tens to thousands or more) of compounds in an efficient manner. Automated and miniaturized HTS assays are particularly preferred. (Houston and Banks, Curr. Opin. Biotechnol. 8:734-740 (1997). HTS assays are designed to identify “hits” or “lead compounds” having the desired inhibitory property, ie; inhibition of the RRF reaction (supra). Modifications are designed to improve the desired property. Chemical modification of the “hit” or “lead compound” is based on the inhibitory capacity of the “hit” on RRF.

There are a number of different libraries used for the identification of specific small molecule inhibitors, including, but not limited to, (1) chemical libraries, (2) natural product libraries, and (3) combinatorial libraries comprised of random peptides, oligonucleotides or organic molecules. These libraries are well known to those of skill in the art.

The invention also provides methods for identifying a therapeutic agent with bactericidal and/or bacteriostatic activity. The methods include, but are not limited to: a) mixing the reaction components (supra) with the inhibitor(s) to be tested; b) measuring the amount of polyribosomes that are converted into monosomes; and c) identifying an inhibitor(s) by a decrease in the conversion of polysomes to monosomes; d) confirming that the inhibitor inhibits translation of natural mRNA; and e) establishing that the inhibitor does not inhibit translation of a synthetic polynucleotide.

Pharmaceutical Compositions

The present invention also provides pharmaceutical compositions. Such compositions comprise a therapeutically effective amount of the compound (an inhibitor of RRF), and a pharmaceutically acceptable carrier or excipient. Such a carrier includes, any material which, when combined with a compound of the invention, allows the compound to retain biological activity, i.e., the ability to inhibit RRF activity, and is non-reactive with the subject's immune system. Examples of acceptable carriers include, but are not limited to: saline, buffered saline, dextrose, water, glycerol, ethanol, and combinations thereof. The carrier and composition can be sterile. The formulation should suit the mode of administration.

Compositions comprising such carriers are formulated by well known conventional methods (see, for example, Remington's Pharmaceutical Sciences, Chapter 43, 14th Ed., Mack Publishing Col, Easton Pa. 18042, USA).

The pharmaceutical compositions are packaged in forms convenient for delivery. The compositions can be enclosed within a capsule, sachet, cachet, gelatin, paper, or other container. These delivery forms are preferred when compatible with entry of the composition into the recipient organism and, particularly, when the composition is being delivered in unit dose form. The dosage units can be packaged, e.g., in tablets, capsules, suppositories or cachets.

Results

The present invention clearly reveals that oligoG inhibits RRF. As the concentration of oligoG, such as the sd-G4, or derivatives thereof, is increased there is a rapid and significant increase in the inhibition of RRF. Inhibiting RRF inhibits the disassembly of the post-termination complex, as seen by the inhibition of the conversion of polyribosomes to monosomes (FIG. 1).

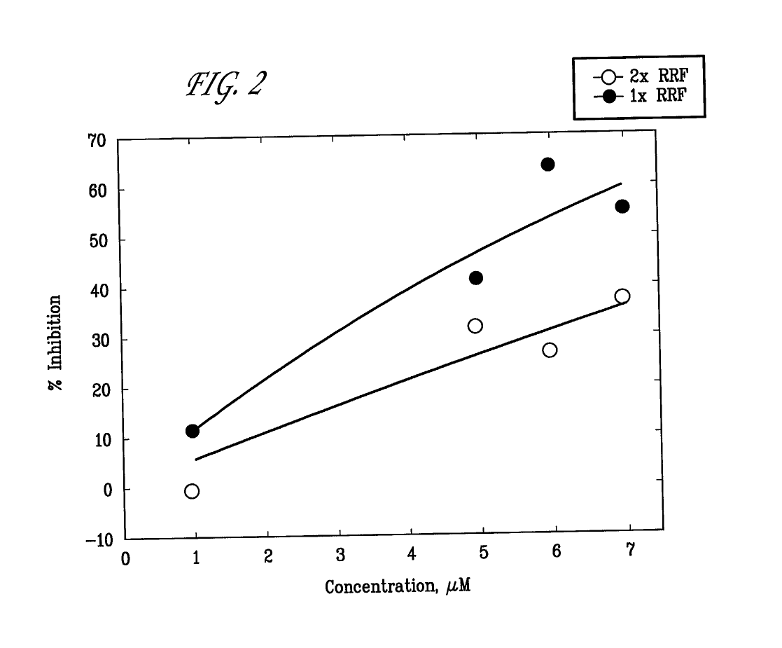

The inhibitory effect of sd-G4 is effectively overcome by higher amounts of RRF (FIG. 2). This direct interaction of sd-G4 with RRF is further revealed in FIG. 9. Incubating labeled sd-G4 with RRF results in the retardation of the electrophoretic mobility of sd-G4 due to the apparent increase of molecular weight and charge of sd-G4 caused by the binding of sd-G4 to RRF. This reaction is specific in that addition of increasing amounts of unlabeled sd-G4 competitively inhibits the binding of the labeled sd-G4, resulting in decreasing amounts of the retarded sd-G4 band. This interaction of sd-G4, or derivatives thereof, with RRF is critical for rational drug design of RRF inhibitors, as sd-G4, or derivatives thereof, will serve as a lead compound for the development of small molecule inhibitors of RRF (supra). Small molecule inhibitors are those that are structurally and functionally related to a lead compound (sd-G4, or derivatives thereof) with improved properties. Further, administration of sd-G4, or derivatives thereof, to mice reveals their lack of toxicity, and thus safety, for use as a therapeutic agent (Table 1). The present invention provides sd-G4, or derivatives thereof, as a lead compound to identify small molecules that inhibit the activity of RRF, thereby inhibiting mRNA dissociation from the polyribosome, with the subsequent inhibition of protein synthesis. This reaction will result in the death of any infecting pathogenic bacteria, as has been previously demonstrated. (Janosi, L., et.al., Adv. Biophys, 32, 121-201, 1996). Crystallization of an sd-G4, or derivatives thereof, and RRF complex will allow for the rational drug design of anti-RRF agents using sdG-4, or derivatives thereof, as a lead compound (supra).

The present invention further reveals that the inhibitory effect of sd-G4, or derivatives thereof, is dependent on natural mRNA. MS 2 phage, a bacterial virus, is used as a source of natural mRNA. As the concentration of sd-G4 is increased, there is a concomitant decrease in the synthesis of protein, as shown in FIG. 3. This inhibition is dependent on the inhibition of RRF, and not EF-G, as sd-G4 does not inhibit translation of a polyuridylic acid (polyU) into polyphenylalanine (FIG. 4). The translation of a synthetic polynucleotide, such as polyU, is dependent on EF-G (elongation factor G), EF-Tu (elongation factor Tu) and ribosomes, but not on RRF. As the RRF reaction is dependent on EF-G, sd-G4 inhibition of EF-G could result in the inhibition of the RRF reaction. However, as the concentration of sd-G4 is increased in the presence of the synthetic polynucleotide polyU, there is a corresponding increase in the incorporation of ¹⁴C-phenylalanine (FIG. 4). This experiment shows that sd-G4 does not inhibit ribosomes or EF-G. Therefore, since RRF is not involved in artificial polypeptide synthesis, lack of an inhibitory effect on the translation of this synthetic polynucleotide by sd-G4 implies that sd-G4 is specific to RRF. Artificial polypeptide synthesis is exemplified herein by the synthesis of polyphenylalinine from a polyuridylic acid. As this is an example of the synthesis of a synthetic polypeptide from a synthetic polynucleotide, it is in no means meant to limit the invention. It is understood by those skilled in the art that the absence of inhibition of RRF by sd-G4, or derivatives thereof, during polyphenylalanine synthesis will also occur in other in vitro polypeptide synthesis systems programmed by synthetic polynucleotides, for example polyadenylic acid. Thus, the present invention reveals that inhibition of protein synthesis by sd-G4 is indeed due to a specific inhibition of RRF.

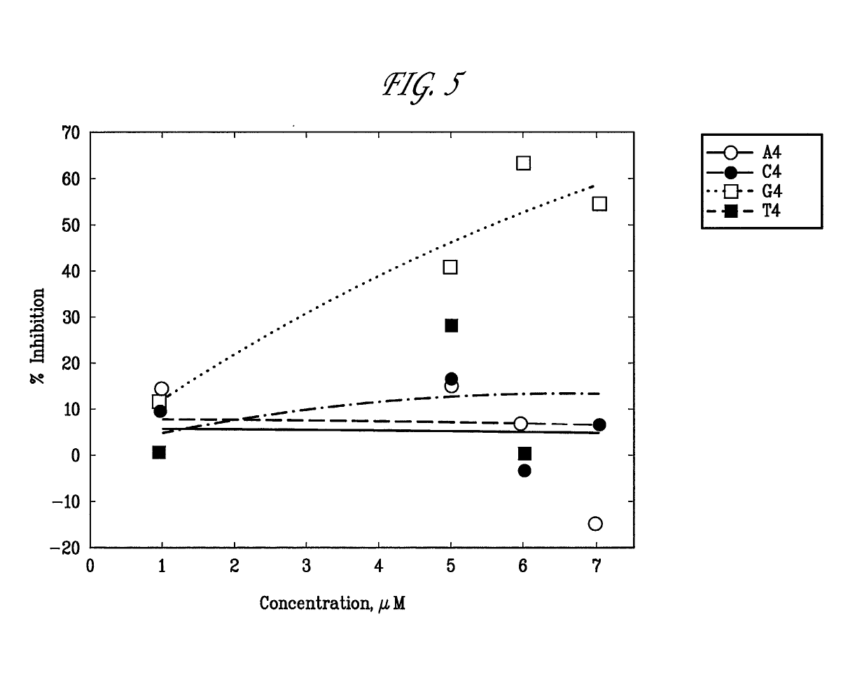

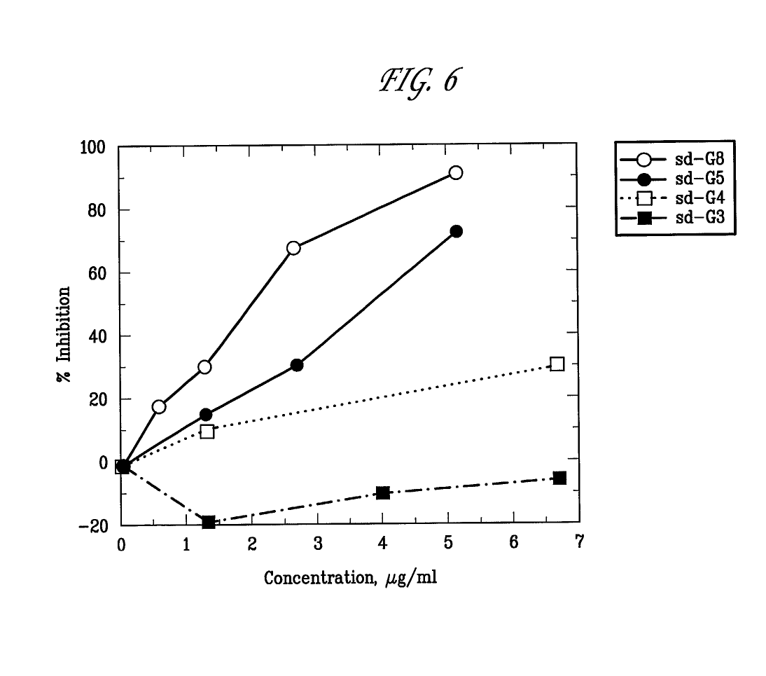

The present invention reveals that the inhibitory effect of the oligoG is dependent on both length and nucleoside composition. The RRF reaction is specifically inhibited by phosphorothioate oligoguanylic acids. Tetraadenylic, tetracytidylic, and tetrathymidylic acids are ineffective in inhibiting RRF (FIG. 5). Further, the inhibitory effect of the oligoG is dependent on length. The minimum effective length of oligoG is 4 nucleotides. Increasing the length up to 8 results in a corresponding increase in inhibitory activity (FIG. 6). While it is possible to utilize all compounds derived from the guanylic acid derivative as lead compounds, it is desirable to have as small a compound as possible for the development of therapeutic agents, as they are more readily taken up by the bacterial cells.

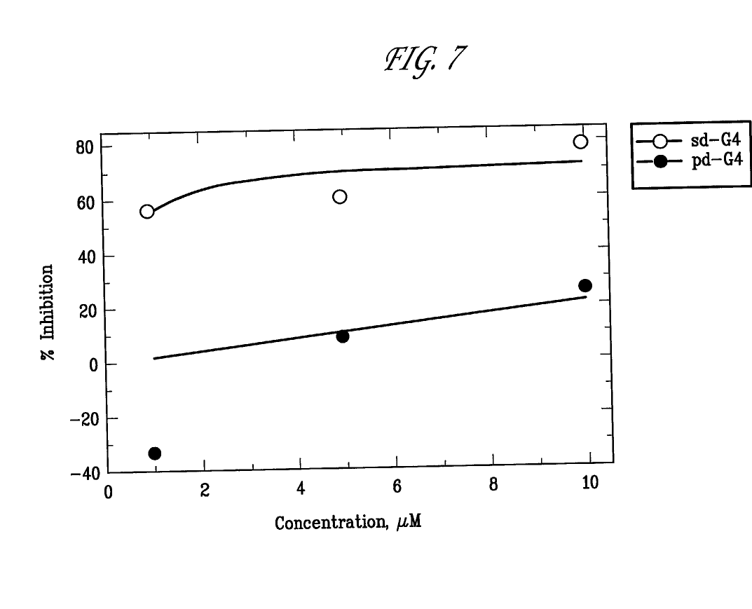

The inhibitory action of RRF by sd-G4 is also dependent on the presence of the phosphorothioate linkage of the nucleotides (FIG. 7). While the phosphodiester oligoG retains the ability to inhibit RRF to some extent, the efficacy of this derivative is significantly less than that of the phosphothioate derivative.

RRF catalyzes the disassembly of the post-termination complex during protein synthesis. The crystal structure of RRF reveals that it is an L-shaped molecule, a near perfect mimic of tRNA, consisting of two domains, domain 1 being reminiscent of the anticodon arm of tRNA. (Selmer, M. et al., Science 286: 2349-2352, 1999; Kim, K. K., et al., EMBO J 19(10): 2362-2370, 2000).

The RRF reaction proceeds in a stepwise manner. (Selmer, M. et al., Science 286: 2349-2352, 1999). The first step is the binding of RRF to the ribosomal A-site. During disassembly of the post-termination complex the RRF translocates from the A-site to the P-site on the ribosome by EF-G and GTP, thereby causing the tRNA to be released from the complex. Following the release of the deacylated tRNA, the ribosomes are released from the mRNA, concomitant with the release of RRF and EF-G, completing the disassembly process. The ribosomes are now free to bind to another mRNA and initiate translation of another polypeptide. The fact that sd-G4 inhibits the disassembly process but does not inhibit the release of deacylated tRNA (FIG. 8) implies that sd-G4 acts at the last step of the disassembly. In other words, sd-G4 does not inhibit the translocation of RRF by EF-G or the disassociation of tRNA from ribosomes. sd-G4 inhibits the step where mRNA disassociates from ribosomes.

sd-G4 binds directly to RRF, as evidenced by the data presented in FIG. 9. The data presented in FIG. 2 provides further evidence to the direct interaction between sd-G4 and RRF, as increasing the amount of RRF reduces the inhibitory effect of sd-G4. This direct interaction of sd-G4 and RRF forms the basis for a rational drug design approach for an efficacious anti-RRF compound. By using the sd-G4 as a lead compound in the development of therapeutic agents that inhibit the dissociation of the ribosomes from mRNA, the present invention will allow for novel antibacterial therapeutic agents to be developed.

For sd-G4 to be active as a specific inhibitor of RRF sd-G4 must be non-toxic in living organisms. Dosages (20-125 mg/kg) of sd-G4 and its related tetra nucleotides are administered to mice intraperitonially, as well as intraveniously, and neither acute nor chronic toxicity is noted, i.e., no grossly observable changes in growth activity, locomotor activity, food intake, or water intake during an observation period of 25 days. Moreover, no pathological signs are noted at autopsy (Table 1). The in vivo data reveal that the effective concentration of sd-G4 which will inhibit bacterial RRF does not show any deleterious effects on the experimental animals. Thus, sd-G4 is best choice for a lead compound in the computer based rational design of the anti RRF agents.

The present invention reveals that oligoG is a specific inhibitor of RRF, thereby allowing it to serve as a lead compound for the development of anti-RRF therapeutic agents. While this invention is described with a reference to specific embodiments, it will be obvious to those of ordinary skill in the art that variations in these methods and compositions may be used and that it is intended that the invention may be practiced otherwise than as specifically described herein. For example, related oligoguanylic acids, including, but not limited to, oligoguanyl ribonucleic acids, exert an identical action on HIV reverse transcriptase. (described more fully in Japanese patent applications: JP 6-200029 to 32, JP 6-238313 to 17, JP 6-92809, and JP 6-92810, incorporated herein by reference). Accordingly, this invention includes all modifications encompassed within the spirit and scope of the invention as defined by the claims.

What is claimed is:

1. A method of identifying an inhibitor of RRF, comprising:
a) mixing said inhibitor in a reaction comprising RRF and polysome;
b) measuring a conversion of said polysome to monosomes;
c) identifying said inhibitor by a decrease in said conversion of said polysome to said monosomes;
d) confirming that said inhibitor inhibits translation of natural mRNA; and
e) establishing that said inhibitor does not inhibit translation of a synthetic polynucleotide.

2. The method of claim 1, wherein said inhibitor is sd-G4, or derivatives thereof.

3. An inhibitor of RRF, comprising a small molecule that is structurally and functionally related to sd-G4, or derivatives thereof, with improved properties that inhibit RRF, thereby inhibiting disassembly of a post-termination complex and thus protein synthesis.

4. The inhibitor of claim 3, wherein said small molecule that is structurally and functionally related to sd-G4, or derivatives thereof, comprises a ribose moiety.

5. An inhibitor of RRF, comprising use of sd-G4, or derivatives thereof, as a lead compound for further development of a therapeutic agent that inhibits RRF activity.

6. A method of identifying an inhibitor of RRF, wherein sd-G4, or derivatives thereof, is used as a lead compound, comprising:
a) screening by affinity to identify a set of ligands;
b) mixing each of said ligands in a reaction comprising RRF and polysome;
c) measuring a conversion of said polysome to monosomes; and
d) identifying said inhibitor by a decrease in said conversion of said polysome to said monosomes;
e) confirming that said inhibitor inhibits translation of natural mRNA; and
f) establishing that said inhibitor does not inhibit translation of a synthetic polynucleotide.

7. The method of claim 6, wherein said inhibitor comprises a small molecule, said small molecule being structurally and functionally related to said lead compound (sd-G4, or derivatives thereof) with improved properties that inhibit RRF, thereby inhibiting disassembly of a post-termination complex and thus protein synthesis.

8. The method of claim 7, wherein said disassembly of said post-termination complex and thus protein synthesis results in a bactericidal and/or bacteriostatic effect.

9. A pharmaceutical composition, comprising a pharmaceutically acceptable carrier and a therapeutically effective amount of an inhibitor of RRF.
